(19) United States
(12) Patent Application Publication
(10) Pub. No.: US 2026/0191479 A1
(43) Pub. Date: Jul. 9, 2026

(54) Smart Wearable Wristband with Flexible Display and Integrated Biometric Sensors

(71) Applicant: Dean Sherzai, Redondo Beach, CA (US)

(72) Inventor: Dean Sherzai, Redondo Beach, CA (US)

(21) Appl. No.: 19/009,924

(22) Filed: Jan. 4, 2025

(51) Int. Cl. A61B 5/00; A61B 5/0205; A61B 5/021; A61B 5/0531; A61B 5/1455; A61B 5/16; A61B 90/00

(52) U.S. Cl. CPC A61B 5/7445; A61B 5/002; A61B 5/0205; A61B 5/165; A61B 5/486; A61B 5/681; A61B 5/7267; A61B 5/7275; A61B 5/7435; A61B 5/7475; A61B 5/02125; A61B 5/0531; A61B 5/14552; A61B 5/4812; A61B 5/4815; A61B 2090/365; A61B 2560/0242; A61B 2562/0214; A61B 2562/0233; A61B 2562/18

(57) ABSTRACT

The present invention pertains to a multifunctional smart wristband device featuring a flexible touchscreen display and integrated biometric sensors designed for continuous health monitoring, stress management, and communication. The device employs advanced optical sensors for non-invasive blood pressure measurement, pulse oximetry for real-time monitoring of oxygen saturation, and a suite of sensors for stress assessment, including electrodermal activity, heart rate variability, sweat, and temperature. A biofeedback mechanism dynamically provides calming visual stimuli on the display and tactile feedback via embedded actuators to mitigate stress. The wristband further integrates Bluetooth connectivity for real-time health data transmission to smartphones and features a built-in camera and microphone for video communication, enhancing its multifunctional capabilities. Customizable display aesthetics and an ergonomic, skin-friendly design ensure continuous usability in various settings.

### BACKGROUND OF THE INVENTION

### Field of Invention

The present invention pertains to the field of wearable health technology, with a particular emphasis on a multifunctional wristband device that integrates a flexible touchscreen display and a range of biometric sensors. This innovation addresses the growing demand for comfortable, non-invasive solutions to monitor vital health metrics such as blood pressure, oxygen saturation, heart rate, and stress levels. Additionally, the invention enhances user experience by incorporating aesthetic customization, communication tools, and stress management functionalities, establishing itself as a versatile health, wellness, and lifestyle accessory.

### BRIEF SUMMARY OF THE INVENTION

The present invention introduces a smart wearable wristband that seamlessly integrates a flexible AMOLED/OLED touchscreen display with advanced biometric sensors to deliver continuous health monitoring and personalized stress management. Key features include non-invasive blood pressure monitoring, pulse oximetry, stress assessment using sweat and heart rate sensors, and an innovative biofeedback mechanism employing visual and tactile stimuli to promote relaxation.

The wristband further incorporates Bluetooth connectivity for real-time health tracking via smartphone applications, as well as a built-in camera and microphone for video communication. Designed with ergonomic and aesthetic flexibility, the device allows for customizable designs and motifs on its display, enhancing its wearability in diverse social and professional settings.

This invention addresses limitations in current wearable health devices by offering a comprehensive, stylish, and user-friendly solution for monitoring vital signs, managing stress, and facilitating communication, all within a single compact wearable device.

### BRIEF DESCRIPTION OF THE FIGURES

FIG. 1.101-1.113: The flowchart outlines the operational sequence of the smart wristband, starting from device activation and sensor calibration, followed by real-time biometric data acquisition and analysis, dynamic biofeedback delivery, and seamless data transmission to external devices, concluding with efficient power management and charging for continuous use.

### DETAILED DESCRIPTION

The present invention relates to a multifunctional wearable wristband device, which combines advanced biometric monitoring technologies, personalized stress management solutions, and customizable aesthetic features into a single, ergonomic wearable platform. The invention seeks to overcome deficiencies observed in existing wearable health technologies by providing a compact, user-friendly device capable of continuous health monitoring, stress alleviation, and seamless integration into various aspects of a user's daily life.

The wristband comprises a flexible AMOLED or OLED touchscreen display, configured to conform to the curvature of the user's wrist. This display serves as the primary interface for accessing health metrics, including but not limited to blood pressure, oxygen saturation levels, heart rate, and stress levels. The flexible display further operates as an aesthetic tool, enabling users to personalize the visual output with dynamic patterns, motifs, and designs that align with their preferences or social settings. The display is laminated with a capacitive touch-sensitive layer, allowing users to interact intuitively with the device while ensuring structural integrity and durability. The flexible display also facilitates the projection of calming visual stimuli as part of a biofeedback mechanism activated in response to elevated stress levels, thus integrating both functional and aesthetic enhancements.

The invention incorporates a novel system for non-inflation blood pressure monitoring. Unlike traditional cuff-based systems, which rely on physical constriction for measurement, the wristband employs optical sensors in combination with pulse wave velocity analysis and other proprietary non-invasive methodologies. These sensors are configured to continuously monitor blood pressure without the need for user intervention or discomfort, thereby ensuring convenience and usability. Data collected by the sensors is processed in real time by an integrated microcontroller unit (MCU) that utilizes advanced algorithms to provide accurate blood pressure estimations, which are displayed on the wristband and transmitted to an external device via Bluetooth connectivity for long-term analysis and record-keeping.

In addition to blood pressure monitoring, the device is equipped with pulse oximetry sensors that measure blood oxygen saturation (SpO₂) levels. The pulse oximetry system operates continuously to monitor respiratory health and performance during physical activities or rest periods. The photoplethysmographic (PPG) sensors integrated into the device also measure heart rate and heart rate variability, providing critical data for both fitness tracking and health diagnostics.

The invention further features an advanced stress monitoring and biofeedback mechanism, which combines inputs from multiple biometric sensors, including electrodermal activity (EDA) sensors, PPG sensors, sweat rate sensors, and skin temperature sensors. These inputs are analyzed using proprietary algorithms to assess the user's physiological and emotional state. In response to detected stress levels, the biofeedback system is activated to provide both visual and tactile stimuli aimed at promoting relaxation. Specifically, the flexible display projects calming animations and patterns, while tactile actuators embedded beneath the display deliver rhythmic pulses or vibrations. The intensity, frequency, and duration of the biofeedback stimuli are dynamically adjusted based on the user's physiological data and pre-programmed preferences, thereby providing a highly personalized stress management experience.

The wristband includes integrated Bluetooth Low Energy (BLE) connectivity, enabling seamless communication with paired smartphones or other external devices. This connectivity facilitates real-time synchronization of health metrics, transmission of alerts, and access to advanced analytics through a companion application. Additionally, the wristband incorporates a built-in camera and microphone, allowing users to engage in video calls or voice interactions directly from the device. The flexible display serves as a dynamic communication interface, displaying notifications, calls, and other relevant data in an organized and aesthetically pleasing manner.

The structural and material design of the wristband is optimized for continuous wear, with hypoallergenic and skin-friendly materials ensuring comfort during prolonged use. The device's lightweight and flexible form factor accommodates a wide range of wrist sizes and movement patterns, making it suitable for both casual and active wear. The modular design of the wristband allows for aesthetic customization, enabling users to select or upload display patterns that align with their personal style or specific occasions.

Power for the wristband is provided by a flexible lithium-polymer (Li-Po) battery integrated into the device. The battery system is managed by a power management integrated circuit (PMIC), which dynamically regulates power consumption to ensure a minimum of 36 hours of continuous operation under typical usage conditions. The PMIC further supports wireless charging via an inductive coil or magnetic connector, offering users flexibility in maintaining the device's operational readiness.

The invention also incorporates sleep monitoring capabilities. The sensors within the device track sleep stages, heart rate, and motion patterns to generate comprehensive insights into the user's sleep quality. These insights are processed by the companion application, which provides actionable recommendations for improving sleep habits. The wristband's tactile actuators can be programmed to deliver gentle wake-up pulses during optimal points in the user's sleep cycle, further enhancing its utility as a holistic wellness tool.

This invention overcomes significant limitations present in existing wearable technologies. It eliminates the need for multiple separate devices by integrating a diverse array of biometric monitoring functionalities into a single compact platform. The non-invasive and ergonomic design ensures user comfort, while the customizable aesthetic features make the device suitable for continuous wear in various social, professional, and athletic contexts. Furthermore, the device's biofeedback capabilities represent a significant advancement in stress management, providing users with real-time tools to address and mitigate the effects of stress on their overall well-being.

The present invention provides a comprehensive solution that combines cutting-edge health monitoring technologies, innovative stress management systems, and aesthetic versatility into a single wearable device. By addressing the shortcomings of prior art, this wristband establishes itself as a transformative innovation in the field of wearable health technology, offering unparalleled value and functionality to its users.

In general, machine learning algorithms are used to make a prediction or classification regard user health using inputs obtained at the wearable device. Based on some input data, which can be labeled or unlabeled, the algorithm will produce an estimate about a pattern in the data.

An error function evaluates the prediction of the model. If there are known examples, an error function can make a comparison to assess the accuracy of the model. A model optimization process then occurs. If the model can fit better to the data points in the training set, then weights are adjusted to reduce the discrepancy between the known example and the model estimate. The algorithm will repeat this “evaluate and optimize” process, updating weights autonomously until a threshold of accuracy has been met.

Supervised learning in particular uses a training set to teach models to yield the desired output. This training dataset includes inputs and correct outputs, which enables the model to learn over time. The algorithm measures its accuracy through the loss function, adjusting until the error has been sufficiently minimized. Thus, through the computer-implemented process described above, the present invention can improve its ability to predict and detect e.g., user health insights about physiologic parameters.

After training, the machine learning categorization engine processes the sensor data using pre-trained models trained on datasets of other users and their health statuses and outcomes. It comprises an application-specific integrated circuit (ASIC) for an artificial neural network connected to the computer memory device, the ASIC comprising: a plurality of neurons organized in an array, wherein each neuron comprises a register, a processing element and at least one input, and a plurality of synaptic circuits, each synaptic circuit including a memory for storing a synaptic weight, wherein each neuron is connected to at least one other neuron via one of the plurality of synaptic circuits, wherein the array is configured to analyze said wearable sensor data algorithms trained on historical datasets associating wearable sensor data with health statuses and/or outcomes, wherein the AI/ML categorization engine makes a prediction regarding health statuses and/or outcomes.

### DETAILED DESCRIPTION OF FIGURES

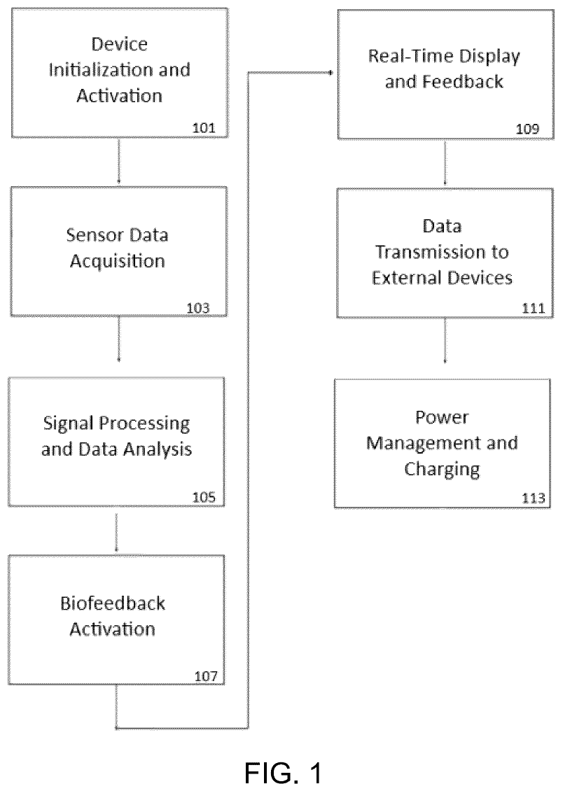

FIG. 1.101: Device Initialization and Activation-The user powers on the wristband by pressing the activation button or tapping the display, triggering the microcontroller (MCU) to initialize system components, including biometric sensors, display drivers, and connectivity modules. During this step, a system check ensures that all sensors, actuators, and communication modules are functional. The device then enters a standby or active monitoring mode based on the user's settings.

FIG. 1.103: Sensor Data Acquisition—The device's integrated sensors, including optical sensors for blood pressure and pulse oximetry, electrodermal activity sensors, sweat sensors, and a skin temperature sensor, begin acquiring biometric data. These sensors are calibrated automatically by the MCU to account for environmental conditions such as ambient light or temperature, ensuring precise data collection.

FIG. 1.105: Signal Processing and Data Analysis—The acquired sensor data is processed by the MCU using advanced algorithms. Optical data from photoplethysmography (PPG) is analyzed for blood pressure and heart rate estimations, while electrodermal and sweat sensor inputs are combined to assess stress levels. The data is cross-referenced to improve accuracy, and health metrics such as oxygen saturation, blood pressure, and stress levels are extracted and stored in memory.

FIG. 1.107: Biofeedback Activation—If elevated stress levels or abnormal health metrics are detected, the wristband's biofeedback system is activated. Visual feedback, such as calming animations, appears on the display, while embedded tactile actuators deliver rhythmic vibrations to encourage relaxation. The intensity and frequency of feedback are dynamically adjusted based on real-time data.

FIG. 1.109: Real-Time Display and Feedback—The processed health metrics are displayed on the flexible AMOLED/OLED screen in a user-friendly format, including graphs and alerts for abnormal readings. Users can interact with the display to toggle between metrics or activate additional features. Simultaneously, tactile notifications or animations provide subtle feedback for stress or health alerts.

FIG. 1.111: Data Transmission to External Devices—The wristband uses Bluetooth Low Energy (BLE) to transmit health data securely to a paired smartphone or other compatible devices. The accompanying app provides detailed analytics, trend tracking, and historical comparisons. The device also supports real-time communication for video calls, with the camera and microphone transmitting data directly to the connected smartphone.

FIG. 1.113: Power Management and Charging—The power management system monitors battery usage, dynamically adjusting energy consumption based on activity levels. The device alerts the user when the battery is low and supports wireless or magnetic charging, ensuring quick and efficient recharging while maintaining operational functionality.

What is claimed is:

1. A wearable wristband device comprising:
a. a flexible OLED/AMOLED touchscreen display configured to conform to the curvature of the user's wrist, wherein said display serves as an interactive interface for the visualization of health data and as a customizable aesthetic tool capable of displaying user-selected designs or motifs;
b. a capacitive touch-sensitive layer integrated into said flexible display, facilitating user interaction via touch gestures;
c. a plurality of biometric sensors including at least optical sensors for continuous non-invasive monitoring of blood pressure using pulse wave velocity, pulse oximetry sensors for measuring oxygen saturation levels, and electrodermal activity (EDA) sensors for assessing stress levels through skin conductance changes;
d. a microcontroller configured to process data from said biometric sensors;
e. an application-specific integrated circuit (ASIC) for an artificial neural network connected to the computer memory device, the ASIC comprising: a plurality of neurons organized in an array, wherein each neuron comprises a register, a processing element and at least one input, and a plurality of synaptic circuits, each synaptic circuit including a memory for storing a synaptic weight, wherein each neuron is connected to at least one other neuron via one of the plurality of synaptic circuits, wherein the array is configured to analyze said wearable sensor data algorithms trained on historical datasets associating wearable sensor data with health statuses and/or outcomes, wherein the AI/ML categorization engine makes a prediction regarding health statuses and/or outcomes;
f. a biofeedback mechanism comprising a combination of visual feedback through said flexible display and tactile sensations delivered via embedded haptic actuators, wherein said biofeedback mechanism dynamically responds to the AI/ML categorization engine prediction;
g. a power supply unit comprising a flexible lithium-polymer battery and a power management system optimized to sustain at least 36 hours of continuous operation;
h. a connectivity module configured to enable wireless communication between the wearable wristband device and an external computing device, wherein said connectivity module comprises a Bluetooth Low Energy (BLE) transceiver for real-time data transmission, firmware updates, and extended functionality; and
i. a built-in camera and microphone integrated into said wearable wristband device, enabling video communication and voice interaction functionalities.

2. The wearable wristband device of claim 1, wherein the flexible display further comprises a laminated structure incorporating an optical bonding process to minimize internal reflections and ensure durability under varying environmental conditions.

3. The wearable wristband device of claim 1, wherein the optical sensors utilize multi-wavelength light sources and photodetectors to measure reflected light from subdermal blood flow for deriving blood pressure and oxygen saturation levels.

4. The wearable wristband device of claim 1, wherein the electrodermal activity (EDA) sensors comprise conductive electrodes positioned on the inner surface of the wristband to detect changes in skin conductivity associated with stress levels.

5. The wearable wristband device of claim 1, wherein the biofeedback mechanism includes an adaptive control system configured to adjust the intensity and frequency of tactile and visual feedback based on user preferences and physiological metrics.

6. The wearable wristband device of claim 1, further comprising an adaptive sleep monitoring system, wherein said system analyzes data from biometric sensors to determine sleep stages and provides recommendations to improve sleep quality via a connected external application.

7. The wearable wristband device of claim 1, wherein the connectivity module further comprises a dynamic communication interface configured to display notifications, messages, and real-time calls on the flexible display.

8. The wearable wristband device of claim 1, wherein the camera and microphone are positioned along the edge of the flexible display, wherein said positioning ensures unobtrusive integration into the wristband's ergonomic structure.

9. The wearable wristband device of claim 1, wherein the power management system includes a wireless charging coil integrated into the wristband, configured to enable inductive charging while maintaining the flexibility of the device.

10. The wearable wristband device of claim 1, wherein the device further comprises a tactile notification system, wherein said system conveys alerts, reminders, or biofeedback responses through haptic actuators embedded beneath the flexible display.

11. The wearable wristband device of claim 1, wherein the flexible display is constructed from a polyimide substrate coated with multiple thin-film encapsulation layers to provide protection against moisture and mechanical stress.

12. The wearable wristband device of claim 1, wherein the device further comprises end-to-end encryption of all data collected, transmitted, and stored, ensuring compliance with global data privacy standards, including GDPR and HIPAA.

13. The wearable wristband device of claim 1, wherein the biometric sensors are configured to operate in a synchronized manner to provide comprehensive health monitoring, including real-time aggregation of blood pressure, oxygen saturation, heart rate, and stress levels.

14. The wearable wristband device of claim 1, wherein the device further comprises modular architecture that facilitates hardware or software upgrades, enabling future feature enhancements without replacing the entire device.

15. The wearable wristband device of claim 1, wherein the wristband incorporates environmental awareness sensors configured to monitor external conditions such as ambient temperature, air quality, and ultraviolet radiation, providing users with actionable data for health-conscious decisions.

16. The wearable wristband device of claim 1, wherein the display further supports augmented reality (AR) functionality, enabling the projection of health education visuals, guided exercises, or enhanced communication interfaces.

17. The wearable wristband device of claim 1, wherein the device is constructed from hypoallergenic and skin-friendly materials, ensuring comfort and durability during prolonged use in various activities, including exercise and sleep.
